(19) United States
(12) Patent Application Publication
(10) Pub. No.: US 2014/0206994 A1
(43) Pub. Date: Jul. 24, 2014

(54) ACCURATE VISUALIZATION OF SOFT TISSUE MOTION ON X-RAY

(71) Applicants: Ameet Kumar Jain, New York, NY (US); Vijay Parthasarathy, Tarrytown, NY (US)

(72) Inventors: Ameet Kumar Jain, New York, NY (US); Vijay Parthasarathy, Tarrytown, NY (US)

(73) Assignee: KONINKLIJKE PHILIPS N.V., EINDHOVEN (NL)

(21) Appl. No.: 14/235,220

(22) PCT Filed: Jul. 26, 2012
(86) PCT No.: PCT/IB2012/053825

§ 371 Date: Jan. 27, 2014

Related U.S. Application Data
(60) Provisional application No. 61/512,931, filed on Jul. 29, 2011.

(51) Int. Cl. A61B 8/08; A61B 6/00

(52) U.S. Cl. CPC A61B 8/5261; A61B 6/469
USPC 600/437

(57) ABSTRACT

A method, system, and program product are provided for accurately visualizing soft tissue motion on an x-ray image. Real time ultrasound images are registered to an x-ray image space. A point of interest is defined. Motion of the selected point is determined from the real time ultrasound images. The determined motion is applied to the selected point on the x-ray image.

### FIELD OF THE INVENTION

The invention relates to the field of medical imaging and more particularly to a method, system and computer program product for accurately visualizing soft tissue motion on an x-ray image with reduced dose by fusing x-ray and ultrasound image data.

### BACKGROUND

X-ray fluoroscopic images are used in various medical interventions for tool guidance and visualization of tools and body structures during a procedure. The x-ray fluoroscopic images provide high resolution tool visualization in real time. However, x-ray images are not particularly adept at detecting soft tissue, such as body structures, or soft tissue motion, such as from breathing, the heart beat, and the like. Also, x-ray fluoroscopic imaging exposes a patient and medical personnel to x-ray dosage, and it is preferable to limit the x-ray dose that a patient or medical personnel receives during an interventional procedure.

Increasingly, 2D/3D ultrasound imaging (U/S) is being used as an aid for guiding cardiac interventions. The key role of U/S is to augment the pre-procedure plan with real time motion information. While U/S can detect soft tissue motion in real time, it does not capture tools well, limiting it's usefulness in tool guidance or visualization.

### SUMMARY

A method, system and program product are provided for accurately visualizing soft tissue motion on an x-ray image.

According to one embodiment, a method is provided for accurately visualizing soft tissue motion on an x-ray image. Real time ultrasound images are registered to an x-ray image space. A point of interest is defined. Motion of the selected point is determined from the real time ultrasound images. The determined motion is applied to the selected point on the x-ray image.

According to one embodiment, multiple points of interest are selected, motion is determined for each selected point, and determined motion for each point is applied to the respective selected points on the x-ray image.

According to one embodiment, the point of interest is selected on x-ray image. According to another embodiment, the point of interest is selected on a 3D model generated from x-ray images. According to another embodiment, the point of interest is selected on the ultrasound image.

According to one embodiment, registering the ultrasound image to the x-ray image comprises electromagnetic tracking of an ultrasound probe in x-ray space.

According to one embodiment, continuous x-ray images are obtained during an intervention procedure. A tool tip used in the procedure is accurately tracked relative to soft tissue on the x-ray stream using a tissue motion overlay from ultrasound tracking.

According to one embodiment, the tracked motion is used to determine a current phase of a cardiac cycle, and the determined phases are used to refine estimates of the motion to more efficiently and accurately track soft tissue motion for overlay on the x-ray images.

According to one embodiment, the x-ray image is automatically zoomed in using the motion overlay to accurately locate the tool in the x-ray image.

According to another embodiment of the present invention, a system is provided for accurately visualizing soft tissue motion on an x-ray image. The system comprises: at least one processor, at least one memory, operably connected to the at least one processor, an ultrasound imaging system operably connected to the at least one processor, and a program of instruction encoded on the at least one memory and executed by the at least one processor to accurately visualizing soft tissue motion on an x-ray image.

According to one embodiment, the program of instruction comprises: program instructions for registering a real time ultrasound images to an x-ray image space, program instructions for defining a point of interest, program instructions for determining motion of the selected point from the real time ultrasound images, and program instructions for applying the determined motion to the selected point on the x-ray image.

According to one embodiment, the system further comprising an x-ray machine, operably connected to the at least one processor, wherein the x-ray machine provides a stream of x-ray images to the at least one processor in real time, and the soft tissue motion is overlaid on each corresponding x-ray image.

According to one embodiment, the system further comprises a surgical tool, wherein, during an intervention procedure, the stream of x-ray images accurately tracks a tip of the tool relative to soft tissue on the x-ray stream using a tissue motion overlay from ultrasound tracking.

According to one embodiment, the x-ray image is automatically zoomed in using the motion overlay to accurately locate the tool in the x-ray stream.

According to another embodiment of the present invention, a computer program product is provided comprising a computer readable storage device having a program of instruction encoded thereon for accurately visualizing soft tissue motion on an x-ray image. The program of instruction comprises: program instructions to register a real time ultrasound images to an x-ray image space, program instructions to define a point of interest, program instructions to determine motion of the selected point from the real time ultrasound images, and program instructions to apply the determined motion to the selected point on the x-ray image.

### BRIEF DESCRIPTION OF THE DRAWINGS

The features and advantages of the invention will be more clearly understood from the following detailed description of the preferred embodiments when read in connection with the accompanying drawing. Included in the drawing are the following figures:

FIG. 1 is an isometric view of a system for accurately visualizing soft tissue motion on an x-ray image according to an embodiment of the present invention;

FIG. 2 is a block diagram of a system for accurately visualizing soft tissue motion on an x-ray image according to an embodiment of the present invention;

FIG. 3 is a flow diagram of a method for accurately visualizing soft tissue motion on an x-ray image according to an embodiment of the present invention;

FIG. 4 is a view of a user interface screen showing selection of a point of interest on an anatomical model according to an embodiment of the present invention;

FIG. 5 is a view of a real time ultrasound image with the point of interest identified according to an embodiment of the present invention;

FIG. 6 is a view of the real time ultrasound image of FIG. 5 showing a motion path for the point of interest; and

FIG. 7 is a view of an x-ray image with the motion path for the point of interest overlaid on the point of interest.

### DETAILED DESCRIPTION

The present invention provides a method, system, and computer program product for accurately visualizing soft tissue motion on an x-ray image. According to one embodiment of the present invention, a real time ultrasound image is registered to an x-ray image. Then, a system user selects a point of interest in one of: the x-ray image, the ultrasound image, or a 3D model of a patients anatomy corresponding to the images. A system tracks movement of the selected point of interest in the ultrasound volume, and calculates the motion path for the selected point. The calculated motion path is then overlaid on the x-ray image.

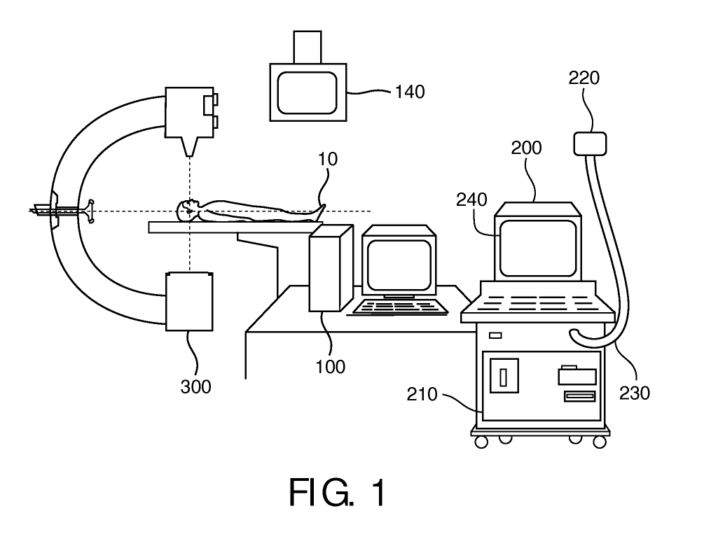

FIG. 1 shows a system for accurately visualizing soft tissue motion on an x-ray image according to an embodiment of the present invention. The imaging system comprises an x-ray machine 300 disposed for taking x-ray imagers of a patient on a table 10. A processing system 100, such as a general purpose computer is operably connected to the x-ray machine and processes x-ray images from the x-ray machine 300. The processed image may be presented on a display 140.

According to one embodiment, the system also comprises an ultrasound system 200 for taking ultrasound images of the patient. The ultrasound system 200 comprises a processing unit 210 for processing ultrasound images, a transducer 220 for generating and receiving sound signals for use in generating ultrasound images. The transducer 220 is connected to the processing unit by a tether 230 which transmits signals between the processing unit 210 and the transducer 220. Ultrasound images may be displayed on a monitor 240. According to an alternative embodiment, the ultrasound images may be processed by the same processing unit 100, which process the x-ray image.

According to one embodiment, the ultrasound images from the ultrasound system 200 are transmitted to the processing system 100. The processing system 100 registers the ultrasound images to the x-ray images from the x-ray machine 300. Then, the processing system 100 receives an indication of a point of interest from a user through a user interface. The processing system tracks the point of interest in an ultrasound volume from the ultrasound images and calculates the path of motion for the point of interest. The processing system overlays the point and path of motion onto the corresponding point in an x-ray image.

FIG. 2 is a block diagram of a system for accurately visualizing soft tissue motion on an x-ray image according to an embodiment of the present invention. The processing system 100 comprises a processor 110 and a memory 120. The processor 110 is operably connected to the memory 120. According to one embodiment, they are connected through a bus 130. The processor 110 may be may be any device capable of executing program instructions, such as one or more microprocessors. The memory may be any volatile or non-volatile memory device, such as a removable disc, a hard drive, a CD, a Random Access Memory (RAM), a Read Only Memory (ROM), or the like. Moreover, the processor 110 may be embodied in a general purpose computer. Moreover, the processor 110 may be embodied in a general purpose computer.

The memory 120 may be any volatile or non-volatile memory device suitable for storing data and program instructions, such as a removable disc, a hard drive, a CD, a Random Access Memory (RAM), a Read Only Memory (ROM), or the like. Moreover, the memory 120 may comprise one or more memory devices.

The processing system 100 may further comprise one or more network connectors 150 for receiving x-ray and ultrasound data. The network connectors may be Uniform Serial Bus (USB) connectors, internet adapters, or any other connector suitable for receiving data from another device, either directly or through a network, such as an intranet or the Internet.

The processing system 100 may also comprise a display 140, such as a monitor for displaying x-ray images, ultrasound images, anatomic models, and the like. One or more monitors may be provided, either in addition to or in place of dedicated monitors for the ultrasound system 200 and for the x-ray machine 300.

Additional input and/or output devices (I/O), such as a keyboard, a mouse, or the like may be provided as part of a user interface to receive indications from a user, such as selection of a point and navigation within an image on the display 140.

The memory 120 has encoded thereon, a program of instruction 121 executable by the processor 110 to accurately visualize soft tissue motion on an x-ray image according to an embodiment of the present invention. The program of instruction 121 comprises: program instructions for registering real time ultrasound images to an x-ray image space 122, program instructions for defining a point of interest 124, program instructions for determining motion of the point of interest on ultrasound images 126, and program instructions for applying the determined motion to the point of interest in the x-ray image 128, which may be different parts of a single application, separate applications callable by each other.

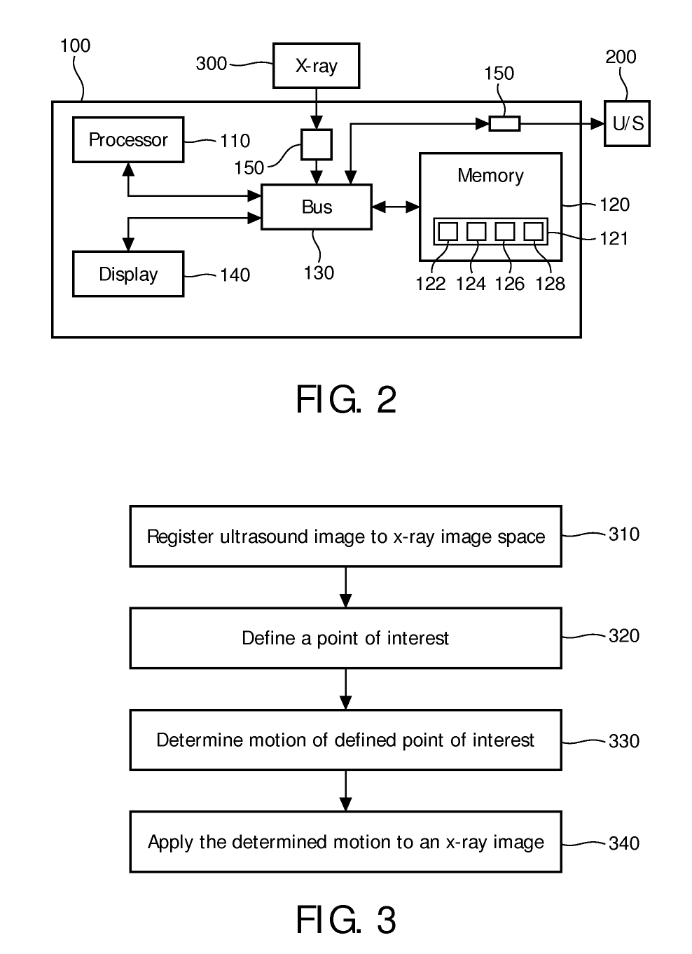

FIG. 3 is a flow diagram of a method for accurately visualizing soft tissue motion on an x-ray image according to an embodiment of the present invention. The program of instruction 121 receives x-ray data from the x-ray machine 300 and generates an x-ray image through a user interface on display 140 as shown in FIG. 4.

The program of instruction 121 further receives ultrasound data from the ultrasound system 200. The ultrasound data may comprise a data stream corresponding to each voxel of a B-mode or radio frequency (rf) image. According to one embodiment, the ultrasound image is a 3D image, however embodiments with 2D ultrasound images are also within the scope of the present invention.

The program of instructions for registering real time ultrasound images to an x-ray image space 122 register the ultrasound images received from the ultrasound system 200 to the image space of the x-ray image received from the x-ray machine 300 (Step 310). The ultrasound images may be registered to the x-ray image space using any of a variety of approaches. These approaches may comprise various combinations of manual alignment, electromagnetic tracking, 2D/3D registration, segmentation, and shape sensing, as well as other techniques. According to one embodiment, the ultrasound probe or transducer 220 is tracked in the x-ray space. For example, one or more sensors may be placed on the ultrasound probe, which are detectable on the x-ray image, thereby providing the 2D location of the ultrasound probe. Moreover, the sensor or sensors may have a pre-determined geometry (size, shape) and/or a pre-determined spacing, which can be used to perform a 2D/3D registration of the x-ray space. Since the 3D location of each voxel of the ultrasound image is known relative to the probe 220, the corresponding coordinates in the x-ray space can be determined by the location of the probe in x-ray space and the 2D/3D registration of the x-ray space.

Alternatively, registration may utilize shape sensing of the tether 230 for the ultrasound probe. That is, Bragg Gratings or Raleigh scatters may be disposed in fiber optic cables in the tether, which are interrogated by light signals to detect local strain, from which local curvatures may be calculated and the shape of the tether determined. The translational and rotational location of the probe 220 may be iteratively calculated from the 2D projection of the tether on the x-ray image and the known 3D tether shape in the form of a transformation matrix. The matrix may then be applied to each voxel of the ultrasound image to determine its corresponding 3D coordinates in the x-ray space.

According to another alternative embodiment, both the ultrasound images and the x-ray image may be registered to the patient table, preoperatively.

The program instructions for defining a point of interest 124 in the program of instructions 121 define a point of interest in the x-ray image, in the ultrasound image, or in a 3D model of the anatomy corresponding to the x-ray image (derived from a pre-procedure CT scan or an intra-operative cone-beam scan, for example, and registered to the x-ray image) (Step 320), as shown in FIG. 4. This may be accomplished, for example, by a user navigating to a point of interest in the relevant image or model with a user input device, such as a mouse and indicating a selection, such as with a mouse click. According to one embodiment, the user may be guided in selecting a point of interest by a pull down menu, a dialog box, or the like.

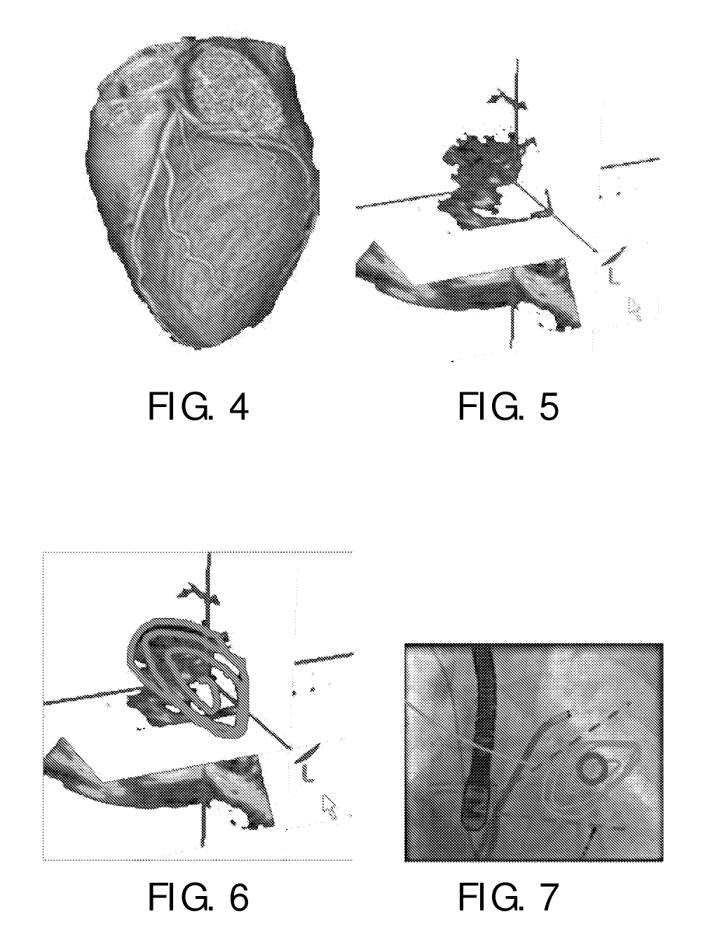

Because the ultrasound image space is registered to the x-ray image space, the defined point of interest may also be located in the ultrasound image space, as shown in FIG. 5. Examples of points of interest include, but are not limited to: ablation points in Afib procedures, the beginning of the coronary ostium in percutaneous aortic valve placements, and other points of surgical interest.

The program instructions for determining motion of the point of interest on ultrasound images 126 determine the real-time motion of soft tissue at the defined point of interest as shown in FIG. 6. That is, the motion of the defined point of interest on the anatomy is tracked in real time on the ultrasound image stream (Step 330). The motion path for the defined point of interest may be determined by matching features in consecutive ultrasound images and subtracting the coordinates for the corresponding voxels of the point of interest using phase signature data in rf or B-mode data.

Alternatively, the motion path for the point of interest may be determined using normalized cross-correlation or sum of squares differences, which are well known in the art, using or any other suitable technique.

The program instructions for applying the determined motion to the point of interest in the x-ray image 128 apply the determined motion (Step 340) from the ultrasound tracking to a live x-ray image as shown in FIG. 5. Thus, the soft tissue motion can be accurately visualized in a real time x-ray image. The 2D x-ray coordinates can be converted into 3D US real time coordinates using a combination of system calibration, reconstruction, and real time tracking.

According to one embodiment of the present invention, multiple points are interest are defined. Then, motion is determined for each point of interest from the ultrasound images, and the motion of each point of interest is overlaid on the real-time x-ray image.

During an intervention procedure, as continuous x-ray images are obtained, a tool tip can be accurately tracked relative to soft tissue on the x-ray steam using a tissue motion overlay from ultrasound tracking. Also, tracked motion can be used to determine a current phase of a cardiac or breathing cycle. The determined phases may then be used to refine estimates of the motion to more efficiently and accurately track soft tissue motion for overlay on the x-ray images.

In another embodiment, the 3D trajectory of a tool is obtained using a biplane system when both of the x-ray streams are obtained simultaneously using two x-ray machines. Motion tracked using ultrasound data is then overlaid on the resulting 3D image space.

In another embodiment, the x-ray image can be automatically zoomed in using the motion overlay to accurately locate the tool in the x-ray image. Thus, dose can be reduced due to the narrower focus of the x-ray.

The invention can take the form of an entirely hardware embodiment or an embodiment containing both hardware and software elements. In an exemplary embodiment, the invention is implemented in software, which includes but is not limited to firmware, resident software, microcode, etc.

Furthermore, the invention may take the form of a computer program product accessible from a computer-usable or computer-readable medium providing program code for use by or in connection with a computer or any instruction execution system or device. For the purposes of this description, a computer-usable or computer readable medium may be any apparatus that can contain or store the program for use by or in connection with the instruction execution system, apparatus, or device.

The foregoing method may be realized by a program product comprising a machine-readable medium having a machine-executable program of instructions, which when executed by a machine, such as a computer, performs the steps of the method. This program product may be stored on any of a variety of known machine-readable medium, including but not limited to compact discs, floppy discs, USB memory devices, and the like.

The medium can be an electronic, magnetic, optical, electromagnetic, infrared, or semiconductor system (or apparatus or device). Examples of a computer-readable medium include a semiconductor or solid state memory, magnetic tape, a removable computer diskette, a random access memory (RAM), a read-only memory (ROM), a rigid magnetic disk an optical disk. Current examples of optical disks include compact disk-read only memory (CD-ROM), compact disk-read/write (CD-R/W) and DVD.

The preceding description and accompanying drawing are intended to be illustrative and not limiting of the invention. The scope of the invention is intended to encompass equivalent variations and configurations to the full extent of the following claims.

1. A method for accurately visualizing soft tissue motion on an x-ray image, comprising the steps of:
registering a real time ultrasound images to an x-ray image space (Step 310);
defining a soft tissue point of interest (Step 320);
determining motion of the selected point from the real time ultrasound images (Step 330);
applying the determined motion to the selected point on the x-ray image (Step 340).

2. The method according to claim 1, wherein, multiple points of interest are selected, motion is determined for each selected point, and determined motion for each point is applied to the respective selected points on the x-ray image.

3. The method according to claim 1, wherein, the point of interest is selected on x-ray image.

4. The method according to claim 1, wherein, the point of interest is selected on a 3D model generated from x-ray images.

5. The method according to claim 1, wherein, the point of interest is selected on the ultrasound image.

6. The method according to claim 1, wherein registering the ultrasound image to the x-ray image comprises electromagnetic tracking of an ultrasound probe in x-ray space.

7. The method of claim 1, further comprising:
during an intervention procedure, obtaining continuous x-ray images; and
accurately tracking a tool tip relative to soft tissue on the x-ray stream using a tissue motion overlay from ultrasound tracking

8. The method of claim 7, further comprising:
using tracked motion to determine a current phase of a cardiac cycle; and
using the determined phases to refine estimates of the motion to more efficiently and accurately track soft tissue motion for overlay on the x-ray images.

9. The method of claim 1, wherein the x-ray image is automatically zoomed in using the motion overlay to accurately locate the tool in the x-ray image.

10. A system for accurately visualizing soft tissue motion on an x-ray image, comprising:
at least one processor (110);
at least one memory (120), operably connected to the at least one processor;
an ultrasound imaging system (200) operably connected to the at least one processor; and
a program of instruction (121) encoded on the at least one memory and executed by the at least one processor to accurately visualizing soft tissue motion on an x-ray image;
wherein the program of instruction comprises:
program instructions for registering a real time ultrasound images to an x-ray image space;
program instructions for defining a soft tissue point of interest;
program instructions for determining motion of the selected point from the real time ultrasound images; and
program instructions for applying the determined motion to the selected point on the x-ray image.

11. (canceled)

12. The system of claim 10, further comprising an x-ray machine, operably connected to the at least one processor, wherein the x-ray machine provides a stream of x-ray images to the at least one processor in real time, and the soft tissue motion is overlaid on each corresponding x-ray image.

13. The system of claim 12, further comprising a surgical tool, wherein, during an intervention procedure, the stream of x-ray images accurately tracks a tip of the tool relative to soft tissue on the x-ray stream using a tissue motion overlay from ultrasound tracking.

14. The system of claim 13, wherein the x-ray image is automatically zoomed in using the motion overlay to accurately locate the tool in the x-ray stream.

15. A computer program product comprising a computer readable storage device having a program of instruction encoded thereon for accurately visualizing soft tissue motion on an x-ray image, the program of instruction comprising:
program instructions to register a real time ultrasound images to an x-ray image space;
program instructions to define a soft tissue point of interest;
program instructions to determine motion of the selected point from the real time ultrasound images; and
program instructions to apply the determined motion to the selected point on the x-ray image.
